(19) United States
(12) Patent Application Publication
(10) Pub. No.: US 2014/0309521 A1
(43) Pub. Date: Oct. 16, 2014

(54) ALIASING CORRECTION IN PCMR IMAGING

(71) Applicant: VasSol Inc., River Forest, IL (US)

(72) Inventor: Kezhou Wang, River Forest, IL (US)

(73) Assignee: VasSol Inc., River Forest, IL (US)

(21) Appl. No.: 14/250,711

(22) Filed: Apr. 11, 2014

Related U.S. Application Data
(60) Provisional application No. 61/810,870, filed on Apr. 11, 2013.

(51) Int. Cl. G01R 33/563; G01R 33/565; G01R 33/48; A61B 5/026; G01R 33/56

(52) U.S. Cl. CPC G01R 33/56316; A61B 5/0263; G01R 33/5608; G01R 33/4828; G01R 33/56509
USPC 600/419

(57) ABSTRACT

In PCMR (phase contrast magnetic resonance) images, velocity aliasing appears when the maximum velocity in the imaging region is larger than the VENC (velocity encoding) value. Without correction, the aliasing will lead to distorted measurements. A method and system for velocity aliasing correction are described in this invention. The aliasing correction can improve the accuracy of flow quantization with the PCMR technique.

### CROSS-REFERENCES TO RELATED APPLICATION

The application claims priority to U.S. Provisional Patent Application No. 61/810,870 filed Apr. 11, 2013, entitled “Aliasing Correction In PCMR Imaging”, the entire contents of which is incorporated herein by this reference.

### FIELD OF INVENTION

The present invention relates to a method and system for velocity aliasing correction and more particularly to an aliasing correction improving the accuracy of flow quantization in flow analyses using phase contrast magnetic resonance imaging (PCMR) in flow sensitive areas (blood vessel, cerebrospinal flow channel).

### BACKGROUND OF THE INVENTION

In flow analyses using phase contrast magnetic resonance imaging (PCMR), flow sensitive areas (blood vessel, cerebrospinal flow channel) and flow insensitive areas (background, stationary tissue) are differentiated by phase shift. Stationary tissue is subtracted, and cancelled out from two acquisitions with bipolar velocity gradients of opposite polarities. The phase shift of the flow sensitive area will have non-zero value, and the phase shift is proportional to the How velocity.

### BRIEF DESCRIPTION OF THE DRAWINGS

FIG. 1 depicts an exemplary system for implementing the described aliasing correction method.

FIG. 2 illustrates an exemplary algorithm for aliasing correction.

### DETAILED DESCRIPTION OF THE DRAWINGS

In flow analyses using phase contrast magnetic resonance imaging (PCMR), flow sensitive areas (blood vessel, cerebrospinal flow channel) and flow insensitive areas (background, stationary tissue) are differentiated by phase shift. Stationary tissue is subtracted, and cancelled out from two acquisitions with bipolar velocity gradients of opposite polarities. The phase shift of the flow sensitive area will have non-zero value, and the phase shift is proportional to the flow velocity. For a bipolar gradient pulse of magnitude G, spins flowing with a constant velocity v in the direction of the gradient will accumulate a phase θ, which can be determined by the following relation:

Θ=γG vτ²

Where γ is the gyromagnetic ratio and τ is the width of each lobe of the bipolar gradient pulse. The above relation thus maps the phase of a spin density function as depicted in a PCMR image to a velocity. If phase shifts due to moving spins were the only source of phase in a phase image, the above relation could be used to calculate flow velocities for each pixel in the image. Inhomogeneities in the applied magnetic field (and other factors), however, can impart background phases to particular spins in the phase image. These background phases can be eliminated by construction two phase images with bipolar gradient pulses of equal magnitude and opposite polarity and then subtracting one phase image from the other. Because the background phases are independent of the bipolar gradient pulses, the result of the subtraction is a phase image with velocity dependent phase information and without the background phases.

For accurate flow measurements, it is desirable for the phase signal to be as high as possible in order to differentiate such signals from phase noise. That is, a high signal-to-noise ratio (SNR) is desired. From the relation between the phase Θ imparted to flowing spins and the magnitude U of the bipolar gradient pulse given above, it is seen that the magnitude of the phase signal increases proportionately with the gradient magnitude G. Increasing G beyond a certain point, however, results in distortion of the phase image due to aliasing. When the velocity of a pixel results in phase exceeding 180 degrees, the phase of that pixel in the phase image is aliased back to a phase between −180 degrees and +180 degrees. For example, the phase shift of +181 degree will be aliased to −179 degrees, since there is no difference between +181 and −179 degrees in the phase domain. Such abasing distortion will occur when the velocity and gradient are related by:

Θ=γG vτ²>180 degree

The spin velocity above which aliasing occurs is referred to as the velocity encoding anti-aliasing limit or VENC (Velocity ENCoding and is given by;

VENC=180/γG vτ²

The VENC is thus the aliasing velocity, the highest velocity that can be unambiguously represented in the PCMR image. As a spin velocity increases above the VENC, the phase increases above 160 degrees, and the corresponding velocity to which the phase is mapped is aliased to a velocity in the opposite direction. This will lead to distorted measurements without corrections.

To facility flow calculation and correction, velocity images are created based on phase images. From the above description, the phase shift is proportional to the velocity, and the velocity can be calculated by v=Θ/(γG vτ²). Applying the formula to each pixel in phase images will result in velocity images. To provide better visual contrast of among flow region and possible aliasing pixels, stationary tissue, the obtained velocity images can be displayed in a color or gray scale. In one embodiment, the color property of every pixel is color coded according to the velocity value at the pixel. In this way, the different pixel intensities which distinguish aliasing pixels from flow and surrounding tissues in the velocity image may also provide the velocity information. In the actual velocity image, the pixel intensity may be color mapped to a range of colors or gray scale mapped to shades of black and white. Thus, in one example, the color change is implemented through mapping the intensity value of a pixel of the velocity image to a triple of R, G, B values. There are many variations in such a color mapping scheme. In one embodiment, a color-map of smooth transition from green to red is used to emphasize the vascular structure. Gray-scale mapping, a special color-map with colors range from black to white, can be used as well, providing a familiar film-like rendering of anatomy structure to the radiologists, Other possibilities of color mapping exist which might be adapted to highlight the different tissues or organs of clinical importance.

Described herein is a method and system that makes the correction for aliasing pixels or aliasing regions for PCMR imaging. The aliasing correction can be made either automatically or manually. Automatic correction involves an automatic segmentation of the flow channel region and the abasing area inside. Manual correction needs interactions between users and the Graphic User Interface (GUI). In both cases, the same principle is applied. Assume the maximum velocity is less than twice of the VENC, the velocity aliasing can be reversely corrected by following formulas:

If (V_{aliasing}<0) Corrected Velocity=2×VENC+V_{aliasing}

If (V_{aliasing}>0) Corrected Velocity=2×VENC+V_{aliasing}

The anti-aliasing process is applying the formulas to all aliasing pixels in each velocity image. If the maximum velocity is higher than twice of the VENC, the appearance of the aliasing will be multiple rings with alternative positive and negative values. In theory, the corrections can be applied iterative. However, the results will not be reliable. The PCMR series should be reacquired with a higher VENC value.

The approach described herein provides identification of the aliasing regions in PCMR images. To make the corrections, the aliasing pixels need to be identified, which in turn needs to identify the flow region of interest (ROI), vessel, CSF aqueduct and etc. From above description, the pixels in aliasing regions have an opposite velocity with their surrounding pixels in the flow ROL. Aliasing pixels in a PCMR image can be detected if two or more neighboring pixels contained within a flow channel border exhibit velocities of different signs. In PCMR images, such pixels exhibit abrupt change and discontinuity, and can be assumed to be aliased.

Described herein includes a method to segment the flow ROI from a region of stationary tissue (RST) and background (air) based on the estimation of noise distribution of PCMR magnitude and phase image. For PCMR magnitude images, images are obtained from different intervals of a cardiac cycle. Due to differences in flow, images contain areas with time-varying magnitude and phase information. To estimate the noise of a magnitude image, the images are normalized so that the square sums are the same. An averaged image is calculated and is compared with all signal images. The spatial average of the average sum of square error equals two times of the magnitude image noise variance. The magnitude image is subsequently divided into three regions: background area (air) with intensity dose to image noise; medium SNR with SNR below a threshold value; and area with high SNR above the threshold value. The noise in a phase image depends on the SNR of the magnitude image. By removing background area, the noise of the RST area of phase image is approximated as a Gaussian distribution. Using the similar method as for the magnitude image, the standard deviation of the noise distribution for phase image can be estimated. Since ideally, tissue area without How should have zero phase difference, the average phase difference in studied images is estimated as the average value of the low phase difference area. The flow ROI can be subsequently segmented.

To reliably classify the flow ROI, and intermediate image may be created. The approach is based on the following observation: since images are at different interval within a cardiac cycle, pixels in flow ROI will show time varying intensities. First, the mean and the standard deviation at each image pixels (except areas marked as background) are estimated. The standard deviation is then compared with the noise level of the image (adjusted with SNR). If two values are similar, then the mean value is chosen as the value of intermediate image at that point. If both mean and the standard deviation are significantly deviate with the image noise level, it is very likely inside a flow ROL. Thus, depending on the difference between the mean value at that pixel and the average phase difference of the image, the maximum or minimum value of the phase difference are selected.

The methods described herein may be combined with the 3D localizer described in U.S. Pat. No. 7,739,090 B2. The classification of the flow ROI is simplified due to the 3D localizer. Using the 3D localizer, the flow ROI will be located. at the center of the PCMR images. If the subject moved during the scanning period, the flow ROI will have some offset from the center. However, the offset is expected to be small, since the subject is constrained in the center of coil. Therefore for automatic method, the search for the flow ROI can be limited in the center region of the PCMR images, and result in fast performance. For manual method, a user needs only look for flow ROI in the center region of the images. Manual interaction can also be involved when automatic method fails to identify the flow ROI or produces an unsatisfied flow ROI. With color coded velocity image, user can redraw the flow ROI along the flow channel boundaries. Once the flow ROI is determined, the aliasing pixels in the ROI can be subsequently identified, and aliasing corrections can he applied to the aliasing pixels. The flow information inside the flow ROI will be adjusted accordingly after abasing corrections.

To summarize, How aliasing appears in PCMR imaging when the velocity is higher than the VENC, it is possible to make correction if the maximum velocity is within the twice of the VENC. The aliasing pixels can be automatically or manually identified, since the aliasing pixels have opposite sign from their adjacent flow pixels. The invention herein includes an algorithm which can automatically segment the flow ROI from PCMR magnitude and phase images, identify the flow aliasing pixels in the flow ROI, and perfoml aliasing corrections. The abasing correction can improve the accuracy of flow quantization with PCMR technique.

### EXEMPLARY EMBODIMENT

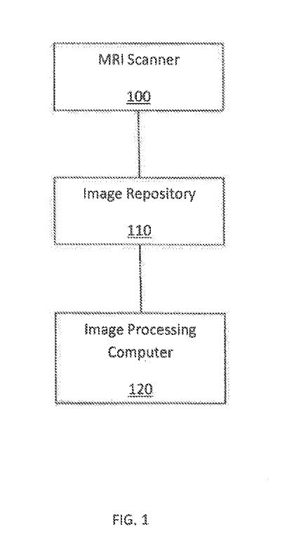

FIG. 1 illustrates an exemplary system that may use to implement the methods described herein. One system component is an MRI scanner 100 that includes a main magnet, gradient coils, RF coils, RF electronics, gradient amplifiers, pulse sequence computer, and an image reconstruction computer. The MR1 scanner generates images and communicates the images to an image repository 110. An image processing computer 120 retrieves selected images from the image repository and is programmed to process the images using methods described herein. The image processing computer may include an input device (e.g., keyboard) and device for displaying the processed images to a user (e.g., a monitor).

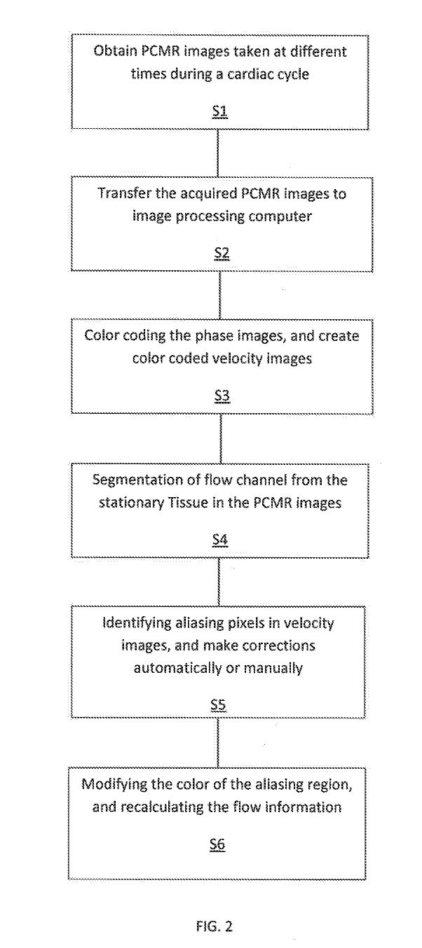

FIG. 2 illustrates an exemplary algorithm that could be executed by the image processing computer and/or MRI scanner. The algorithm may be implemented as a fully automatic process or certain steps may be performed semi-automatic with user interventions. At step S1, PCMR images taken at different times during a single cardiac cycle are obtained, where the PCMR images contain one or more flow regions of interest and are generated as a series of pairs of PCMR images with each member of a pair being generated using a bipolar velocity gradient of opposite polarity to the other member of the pair. Magnitude images and phase images are gem. rated from the PCMR images. At step S2, the magnitude and phase images are transferred to the image processing computer. At step S3, velocity images are created based on the phase images. Then, the phase images are color coded based on the maximum and minimum phase shifts, and the velocity images are color coded based on the calculated maximum and minimum velocities. At step S4, phase images are segmented into one high SNR (signal-noise-ration) region and one or more RSTs (region of stationary tissue). The high SNR region is the candidate flow channel. The border of flow channel is extract automatically or manually if the automatic method fails. The abasing checking in the flow channel is perfomled at the step S5. Following actions are taken; (a) A determination is made whether or not aliasing is present by detecting if velocity discontinuities exist between two or more neighboring pixels located within the border of flow channel of the PCMR image. Aliasing is assumed to be present if any pixels within the channel have velocities in opposite directions and a magnitude difference equal to or greater than the VENC. (b) If aliasing is present, the corrected maximum peak velocity in the PCMR image is computed from a pixel in the PCMR image whose phase is maximally abased. (c) Each aliasing pixel in the velocity image is adjusted. with formula: v correct=2×VENC+v_{aliasing} for negative abasing, or formula V_{correct}=2×VENC+V_{aliasing} for positive abasing, since the aliasing can happen in both directions. In step S6, the color coding of the velocity image is updated with the adjusted maximum and minimum velocities by abasing correction in step S5. The flow rate, velocity and related information will be updated automatically after the corrections.

The invention has been described in conjunction with the foregoing specitlc embodiments. It should be appreciated that those embodiments may also be combined in any manner considered to be advantageous. Also, many alternatives, variations, and modifications will be apparent to those of ordinary skill in the art. Other such alternatives, variations, and modifications are intended to fall within the scope of the following appended claims.

What is claimed is:

1. A method, comprising:
Acquiring PCMR (phase contrast magnetic resonance) images at different times during a single cardiac cycle using a bipolar gradient;
Generating velocity images based on phase images, and color coding the phase and velocity images;
Segmenting the flow region of interest from stationary tissues and background based on the estimation of noise distribution of PCMR magnitude, phase images and velocity images;
Identifying the aliasing pixels in the flow region of interest by comparing the pixel values with surrounding pixels since aliasing pixels have the opposite sign;
Correcting the velocity aliasing using following formulas:
If V_{aliasing}<0) Corrected Velocity=2×VENC+V_{aliasing}
If V_{aliasing}>0) Corrected Velocity=2×VENC+V_{aliasing}
Updating and displaying the color coded phase and velocity images after aliasing corrections; and
Recalculating flow measurements after aliasing corrections.

2. The method of claim 1, further including:
estimating the noise of magnitude images by using an averaged image and the average sum of square error;
Calculating the signal-to-noise ratio;
dividing the images into 3 regions: background with intensity close to image noise, medium signal-to-noise ratio with signal-to-noise ratio below a threshold;
detecting an area with higher signal-to-noise ratio above the threshold value;
Estimating a phase noise in a phase image based on the signal-to-noise ratio of the magnitude image by removing background area;
wherein the noise of region of stationary tissue area of phase image is approximated as a Gaussian distribution to calculate the standard deviation of the noise distribution for estimating phase image; can be estimated; and
Segmenting flow region of interest from the region of stationary tissue, since tissue area without flow should have zero phase difference, and variance close to the noise level, and the flow region has higher phase difference and higher signal-to-noise ratio.

3. A system, comprising:
a magnetic resonance imaging scanner;
an image processing computer configured to receive images generated by the magnetic resonance imaging scanner;
a software program for the image processing computer for obtaining phase contrast magnetic resonance images at different times contained in the flow region of interest and for obtaining the magnitude of the images;
a series of pairs of PCMR images with each member of a pair generated by using a bipolar velocity gradient of opposite polarity to the other member of the pair;
velocity images generated from the PCMR images;
Color coding both the phase and velocity images to increase contrast among pixels in the flow region of interest for determining stationary tissues, background and possible abasing regions;
a magnitude noise level estimated by computing a standard deviation of a pixel intensity in one or more of the magnitude images;
Segment the magnitude images in accordance with pixel magnitude intensity into background regions and into high signal-to-noise ratio regions having the pixel magnitude intensities above a specified threshold;
Segment the phase difference images into region of stationary tissue and flow regions of interest based on signal-to-noise ratio of the magnitude images;
estimate the standard deviation of the noise distribution for phase image Wherein the noise of the region of stationary tissue areas of phase image is approximated as a Gaussian distribution and wherein region of stationary tissue without flow is generally a zero phase difference and a variance close to the noise level and the flow region of interest includes a higher phase difference and a higher signal-to-noise ratio; and
identify the abasing pixels in the flow region of interest to provide abasing correction to each of the aliasing pixels wherein an update of the flow measurements based on the aliasing correction is obtained and wherein an update of the velocity and phase images is obtained on which the aliasing exist.
